(19) United States
(12) Patent Application Publication
(10) Pub. No.: US 2019/0262333 A1
(43) Pub. Date: Aug. 29, 2019

(54) SUSTAINED RELEASE BUPRENORPHINE MICROSPHERES (SRBM) AND METHODS OF USE THEREOF

(71) Applicant: BioDelivery Sciences International, Inc., Raleigh, NC (US)

(72) Inventors: Niraj Vasisht, Cary, NC (US); Andrew Finn, Raleigh, NC (US)

(73) Assignee: BioDelivery Sciences International, Inc., Raleigh, NC (US)

(21) Appl. No.: 16/292,136

(22) Filed: Mar. 4, 2019

Related U.S. Application Data
(63) Continuation of application No. 15/441,197, filed on Feb. 23, 2017.
(60) Provisional application No. 62/298,777, filed on Feb. 23, 2016.

(51) Int. Cl. A61K 31/485; A61K 9/16; A61K 9/00

(52) U.S. Cl. CPC A61K 31/485; A61K 9/1617; A61K 9/1635; A61K 9/0019; A61K 9/1647

(57) ABSTRACT

An autonomous mobile object is configured to transport a plurality of packages having recipients residing in a designated area. The autonomous mobile object includes an operation controller configured to control the autonomous mobile object such that the autonomous mobile object autonomously travels to a designated place associated with the designated area, a storage apparatus having a plurality of compartments, each compartment configured to be independently lockable and unlockable and store a package, and a loading unit configured to load the storage apparatus storing the package and unload the storage apparatus at the designated place.

### CROSS-REFERENCE TO RELATED APPLICATIONS

The application claims priority to Japanese Patent Application No. 2018-031078 filed on Feb. 23, 2018, which is incorporated herein by reference in its entirety including the specification, drawings and abstract.

### BACKGROUND

### 1. Technical Field

The present disclosure relates to an autonomous mobile object, a delivery system configured to deliver a package by using the autonomous mobile object, and a server apparatus.

### 2. Description of Related Art

Studies have been conducted on providing delivery services by using mobile objects capable of autonomous driving. For example, Japanese Patent No. 6164599 (JP 6164599 B) discloses a system for delivering a package to a designated delivery locker through autonomous driving and storing the package in the delivery locker without manual labor.

In the system disclosed in JP 6164599 B, unmanned delivery to a pre-arranged delivery locker as a delivery destination is possible. However, the system may be used only at places where a delivery locker is pre-arranged. Since delivery lockers tend to be installed at places where demand is expected, mainly, a mid- or large-sized multi-family housing or apartment complex, it is difficult for residents living in a single-family house or a small-sized apartment complex to use delivery lockers. Furthermore, even when a delivery locker is pre-arranged, if it is not compatible with or correspond to the system as exemplified, it is not possible to use an unmanned delivery service.

The present disclosure provides an autonomous mobile object, a delivery system configured to deliver a package by using the autonomous mobile object, and a server apparatus used in the delivery system.

An autonomous mobile object according to a first aspect of the present disclosure is configured to transport a plurality of packages having recipients residing in a designated area. The autonomous mobile object includes an operation controller configured to control the autonomous mobile object such that the autonomous mobile object autonomously travels to a designated place associated with the designated area; a storage apparatus having a plurality of compartments, each compartment configured to be independently lockable and unlockable and store a package; and a loading unit configured to load the storage apparatus for storing the packages and to unload the storage apparatus at the designated place.

The autonomous mobile object is a mobile object that autonomously moves on roads. The autonomous mobile object may be an autonomous vehicle that is a mobile object having the objective of moving primarily on roads. The autonomous mobile object is configured to move while the storage apparatus is loaded thereon.

The storage apparatus is a locker having a plurality of compartments, which is a delivery locker, each compartment configured to be independently lockable and unlockable and store a package. The autonomous mobile object may load the storage apparatus by using the loading unit and unload the storage apparatus at a place after the movement. In other words, the autonomous mobile object according to the first aspect of the present disclosure may install the storage apparatus at the designated place by performing an autonomous movement and then unloading the storage apparatus.

According to the first aspect of the present disclosure, a plurality of packages having recipients residing in a designated area is stored in a storage apparatus corresponding to the area, and the autonomous mobile object transports the storage apparatus to a place associated with the corresponding area and unloads the storage apparatus. The place, to which the storage apparatus is transported, may be, for example, a public space, such as inside a railway station, or private property, such as commercial facilities. According to said configuration, since the delivery locker itself may be loaded in the autonomous mobile object and installed at a designated place, a user who would like to receive a package in a designated area may use the unmanned delivery service without limitation.

In addition, the loading unit may load a plurality of storage apparatuses corresponding to a plurality of areas and unload each of the plurality of storage apparatuses at a corresponding place.

As described above, as the loading unit loads a plurality of storage apparatuses for respective assigned areas and unloads each of the storage apparatuses at a corresponding place, the delivery service may be provided across a plurality of places.

Moreover, the storage apparatus may be connected to a fixing unit configured to prevent movement of the storage apparatus from the designated place.

For example, it is possible to prevent theft of the storage apparatus by connecting the storage apparatus to an anchor fixed to a road surface or a building. The fixing unit may be configured to be lockable.

The storage apparatus may unlock, based on the result of authentication on a terminal associated with a recipient, the compartment associated with the recipient.

Furthermore, the storage apparatus may include an autonomous aerial object configured to transport, based on the result of authentication on a terminal associated with a recipient within a designated range, a package stored in the compartment associated with the recipient, in the designated range.

Also, a delivery system according to a second aspect of the present disclosure includes a server apparatus and one or more autonomous mobile objects for transporting a package and is configured to transport a plurality of packages having recipients residing in a designated area. The autonomous mobile objects include an operation controller configured to control the autonomous mobile object such that the autonomous mobile object autonomously travels based on a designated operation command, and a loading unit configured to load a storage apparatus for storing the packages and unload the storage apparatus based on the operation command. The server apparatus is configured to generate the operation command to make the autonomous mobile object travel to a designated place associated with the designated area and to make the loading unit unload the storage apparatus at the designated place, and transmit the operation command to the autonomous mobile object.

As described above, the second aspect of the present disclosure relates to a system that further includes a server apparatus for transmitting an operation command to the autonomous mobile object. The operation command includes, for example, information on a destination or a traveling route, or information on services provided on the route. For example, the operation command may be a command to make the autonomous mobile object transport a package, or install or retrieve a storage apparatus at a designated place based on a traveling route or a destination. The operation command is generated based on, for example, a current position of the autonomous mobile object, the status of the autonomous mobile object, the installation place of the storage apparatus, or the delivery destination of a package stored in the storage apparatus.

Also, the storage apparatus includes a plurality of compartments, each compartment configured to be independently lockable and unlockable. The server apparatus may store information on the recipient and transmit information to unlock a compartment corresponding to the recipient among the compartments of the storage apparatus to an apparatus associated with the recipient.

For example, the server apparatus transmits to the recipient, authentication information such a password, a barcode, or other electronic information to unlock the compartment corresponding to the recipient such that only the rightful recipient may receive the package.

In addition, the server apparatus checks whether packages stored in a storage apparatus have been picked up, and when the pick-up status of the packages satisfies a designated condition, the server apparatus may generate an operation command to make the autonomous mobile object retrieve the storage apparatus and transmit the generated operation command to the autonomous mobile object.

After the packages stored in the storage apparatus are picked up, the autonomous mobile object may retrieve the storage apparatus. The designated condition may be, but is not limited to, for example, when a designated amount of time has passed from the installation of the storage apparatus, or when the number of packages stored is less than a designated number.

Further, an autonomous mobile object according to a third aspect of the present disclosure is configured to transport a plurality of packages having recipients residing in a designated area. The autonomous mobile object includes an operation controller configured to control the autonomous mobile object such that the autonomous mobile object autonomously travels to a designated place associated with the designated area, and a loading unit configured to load a storage apparatus having a plurality of compartments, each compartment configured to be independently lockable and unlockable and store a package, and to unload the storage apparatus at the designated place.

Further, a server apparatus according to a fourth aspect of the present disclosure is used in a delivery system configured to transport a plurality of packages having recipients residing in a designated area. The server apparatus includes a processor configured to generate an operation command for one or more autonomous mobile objects transporting packages, and transmit the generated operation command. The autonomous mobile object includes an operation controller configured to control autonomous driving of the autonomous mobile object, and a loading unit configured to load or unload a storage apparatus storing the packages. The operation command includes a command to make the autonomous mobile object travel to a designated place associated with the designated area and to make the loading unit unload the loaded storage apparatus at the designated place.

The loading unit may load a plurality of the storage apparatuses corresponding to a plurality of areas, and the operation command may include a command to make the loading unit unload each of the plurality of storage apparatuses at a corresponding place.

The storage apparatus may have a plurality of compartments, each compartment configured to be independently lockable and unlockable, and the server apparatus may store information on a recipient, and transmit information to unlock a compartment corresponding to the recipient among the compartments of the storage apparatus to an apparatus associated with the recipient.

The processor may acquire the pick-up status of a package stored in the storage apparatus, and when the pick-up status of the package satisfies a designated condition, generate an operation command to make the autonomous mobile object retrieve the storage apparatus.

Also, another aspect of the present disclosure may be specified as an autonomous mobile object or a delivery system including at least part of the above-described features. Furthermore, the present disclosure may be specified as a method performed by the autonomous mobile object or the delivery system. The above-described process or features may be freely combined and implemented within a range in which no technical contradiction arises.

With each aspect of the present disclosure, it is possible to improve the delivery efficiency of a system for delivering a package using an autonomous mobile object.

### BRIEF DESCRIPTION OF THE DRAWINGS

Features, advantages, and technical and industrial significance of exemplary embodiments will be described below with reference to the accompanying drawings, in which like numerals denote like elements, and wherein:

FIG. 1 is a conceptual diagram of a delivery system according to a first embodiment;

FIG. 2 illustrates the exterior of a storage apparatus;

FIG. 3 is a block diagram schematically illustrating an example of constituent elements of the delivery system;

FIG. 4 illustrates the exterior of an autonomous vehicle;

FIG. 5 illustrates an example of fixing the storage apparatus;

FIG. 6 illustrates an example of operation command data generated by a server apparatus;

FIG. 7 illustrates the flow of data transmitted and received between constituent elements of the system;

FIG. 8 is a flowchart showing a process performed by the autonomous vehicle; and

FIG. 9 illustrates the exterior of a storage apparatus according to a second embodiment.

### DETAILED DESCRIPTION

### First Embodiment

System Overview

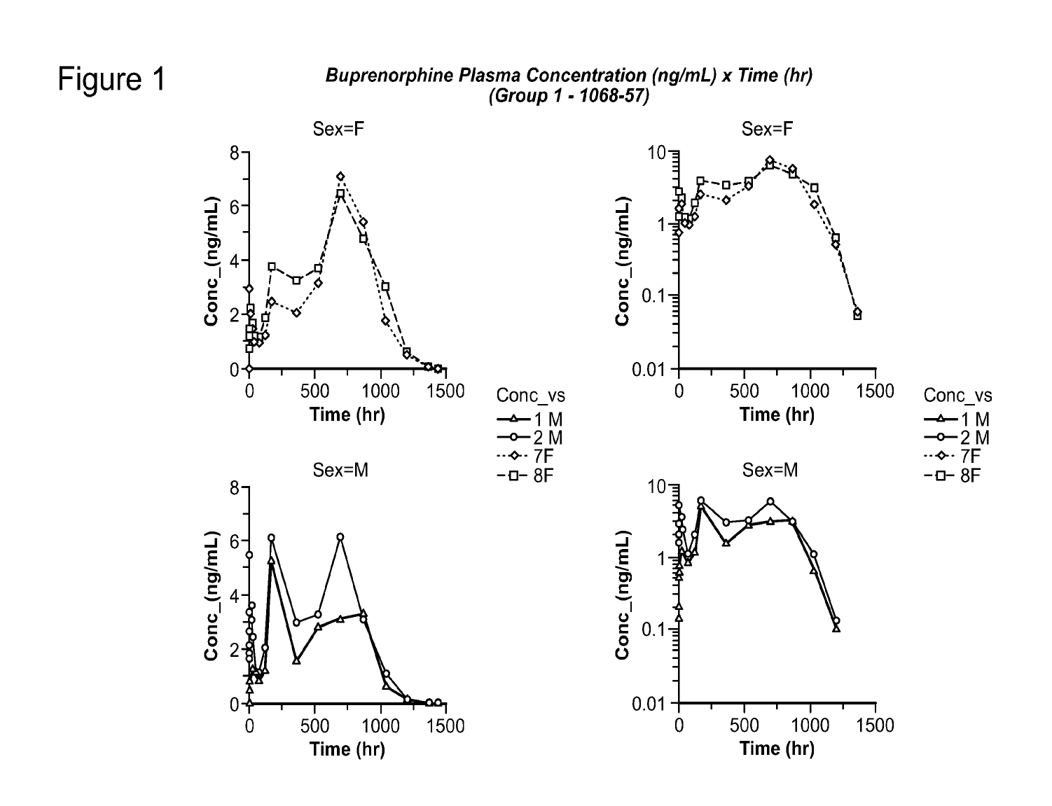

An overview of a delivery system according to a first embodiment will be described with reference to FIG. 1. The delivery system according to the present embodiment includes a plurality of autonomous vehicles 100A, 100B, to 100n that performs autonomous driving based on a given command, a plurality of storage apparatuses 200A, 200B, to 200n that is movable while being loaded on respective autonomous vehicles, and a server apparatus 300 that issues the command. The autonomous vehicles 100 are autonomous driving vehicles that are respectively loaded with the storage apparatuses 200 and provide a delivery service. The server apparatus 300 manages the plurality of the autonomous vehicles 100. In the following description, when the autonomous vehicles are referred to collectively, not individually, the autonomous vehicles will be simply referred to as the autonomous vehicle 100. Furthermore, when the storage apparatuses are collectively, not individually, referred to, the storage apparatuses will be simply referred to as the storage apparatus 200.

The autonomous vehicle 100 is an autonomous driving vehicle capable of loading the storage apparatus 200 thereon and traveling. The autonomous vehicle 100 may also be referred to as an electric vehicle (EV) palette. The autonomous vehicle 100 does not need to be an unmanned vehicle. For example, a sales person, a customer service agent, or a security guard may board the autonomous vehicle 100. Furthermore, the autonomous vehicle 100 does not necessarily need to be a vehicle capable of completely autonomous driving. For example, the autonomous vehicle 100 may be driven or assisted in driving by a person depending on the situation. The autonomous vehicle 100 may load and unload the storage apparatus 200 at a designated place.

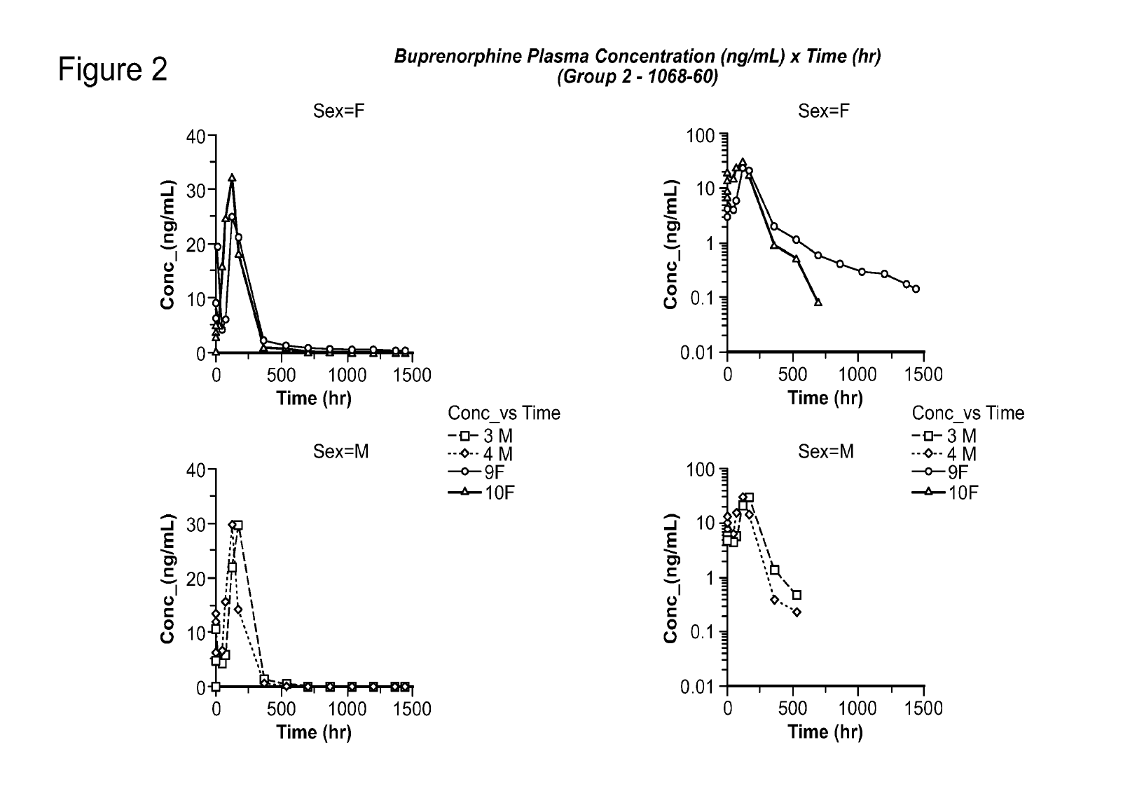

The storage apparatus 200 is a locker-type apparatus having a plurality of compartments, which is a delivery locker, each compartment capable of storing a package. FIG. 2 illustrates the exterior of the storage apparatus 200. As illustrated, the storage apparatus 200 is provided with a plurality of doors, and each of compartments of the storage apparatus 200 is configured to be accessible via a corresponding one of doors. A recipient may unlock a designated compartment through an interface (an input and output unit 204) provided on the storage apparatus 200. A detailed operation thereof will be described hereinbelow.

In the present embodiment, at the collection and delivery base, a plurality of packages with delivery destinations in a certain area are collectively stored in the storage apparatus 200 that corresponds to the area. Each storage apparatus 200 is loaded in a locked state on a corresponding autonomous vehicle 100. Furthermore, the autonomous vehicle 100 loaded with the storage apparatus 200 travels to the corresponding area, and unloads and installs the storage apparatus 200 at a designated place corresponding to the area, for example, at the site of a public facility or a commercial facility. With the above configuration, even recipients who do not have a delivery locker at home may receive packages using a delivery locker.

The server apparatus 300 provides the autonomous vehicle 100 with instruction on the operation thereof. For example, the server apparatus 300 generates an operation command to deliver the storage apparatus 200 from the collection and delivery base to a designated place based on information on the storage apparatus 200 loaded on the autonomous vehicle 100, information on packages stored in the storage apparatus 200, and the like. Moreover, the operation command is not only for traveling. For example, the operation command may include commands to“unload a storage apparatus at a designated place,” “install a storage apparatus at a designated place,” or “retrieve a storage apparatus installed at a designated place.” As such, the operation command may include actions to be taken by the autonomous vehicle 100 other than traveling. Also, the autonomous vehicle 100 may be configured to perform the actions.

System Configuration

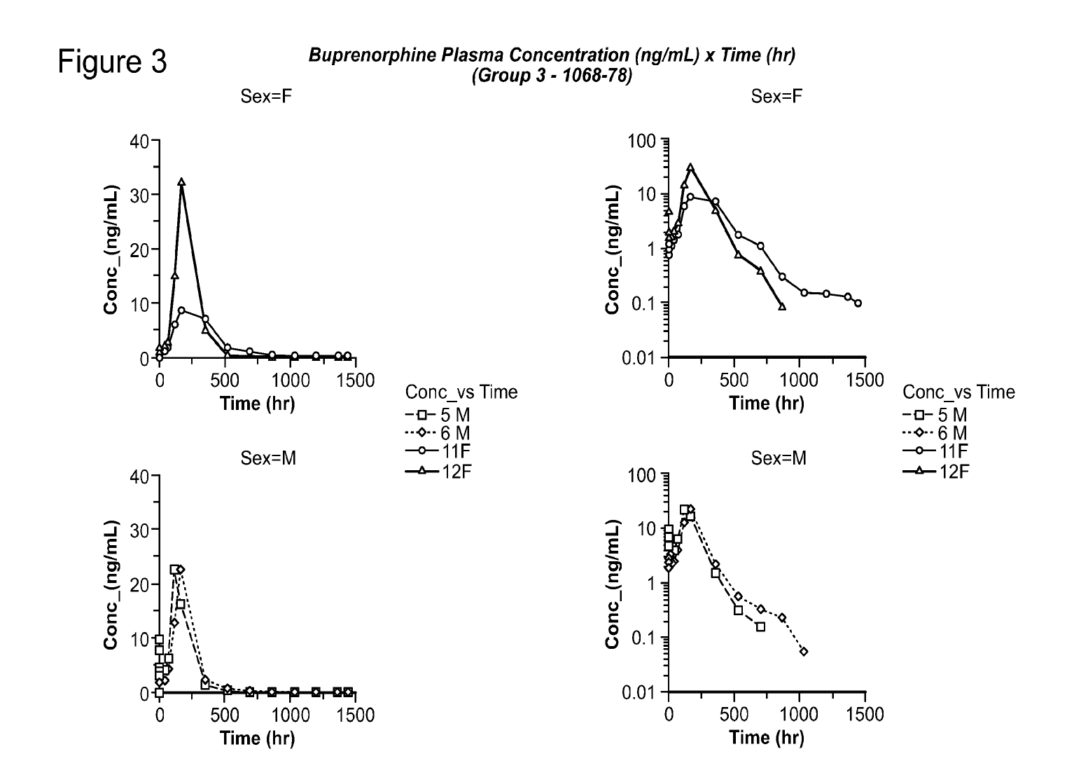

Constituent elements of the system will be described in detail. FIG. 3 is a block diagram schematically illustrating one example of the autonomous vehicle 100, the storage apparatus 200, and the server apparatus 300 that are illustrated in FIG. 1. Further, although FIG. 3 illustrates one autonomous vehicle 100, the autonomous vehicle 100 may include a plurality of autonomous vehicles. Similarly, the storage apparatus 200 loaded on the autonomous vehicle 100 may be a plurality of storage apparatuses.

The autonomous vehicle 100 travels according to the operation command acquired from the server apparatus 300. In particular, the autonomous vehicle 100 generates a traveling route based on the operation command acquired through wireless communication, and travels on a road in an appropriate manner while sensing the surroundings of the vehicle.

The autonomous vehicle 100 includes a sensor 101, a position information acquisition unit 102, a control unit 103, a driving unit 104, a communication unit 105, and a loading unit 106. The autonomous vehicle 100 is operated by power supplied from a battery which is not illustrated.

The sensor 101 senses the surroundings of a vehicle and typically includes a stereo camera, a laser scanner, a light detection and ranging (LIDAR), a radar, or the like. The information acquired by the sensor 101 is transmitted to the control unit 103. The sensor 101 includes a sensor for supporting autonomous driving of the vehicle. The sensor 101 may include a camera mounted on the vehicle body of the autonomous vehicle 100. For example, the sensor 101 may include an imaging device using an image sensor, such as a charge-coupled device (CCD), a metal-oxide-semiconductor (MOS), or a complementary metal-oxide-semiconductor (CMOS). A plurality of cameras may be installed at a plurality of places on the vehicle body. For example, cameras may be respectively installed at the front, rear, right, and left sides of the vehicle body.

The position information acquisition unit 102 acquires the current location of the vehicle and typically includes a GPS receiver or the like. The information acquired by the position information acquisition unit 102 is transmitted to the control unit 103.

The control unit 103 is a computer, which is an example of an operation controller in the present disclosure, configured to control the autonomous vehicle 100 based on the information acquired by the sensor 101. The control unit 103 is, for example, a microcomputer.

The control unit 103 has, as functional modules, an operation plan generation unit 1031, a surroundings detection unit 1032, and an task control unit 1033. Each functional module may be implemented by executing a program stored in a storage, such as a read only memory (ROM), by a central processing unit CPU (neither shown).

The operation plan generation unit 1031 acquires an operation command from the server apparatus 300 and generates an operation plan of a vehicle. In the present embodiment, the operation plan is data defining a route along which the autonomous vehicle 100 travels and a process to be performed by the autonomous vehicle 100 in a part of or the whole of the route. Examples of the data included in the operation plan will be described below.

(1) Data Representing a Route along Which a Vehicle Travels as a Set of Road Links

A route along which a vehicle travels may be automatically generated based on a given starting point and a given destination, for example, by referring to map data stored in the storage which is not shown. In addition, the route along which the vehicle travels may be generated by using an external service.

(2) Data Representing a Process to be Performed by a Vehicle at a Designated Point on a Route

A process to be performed by a vehicle on a route includes, but is not limited to, for example, “unloading the storage apparatus 200” or “retrieving the storage apparatus 200.” For example, the process to be performed by the vehicle on the route may include “transmitting a notification to a person in charge of a store responsible for installing the storage apparatus 200 so as to install the unloaded storage apparatus 200 at a designated place.” The operation plan generated by the operation plan generation unit 1031 is transmitted to the task control unit 1033 that will be described hereinbelow.

The surroundings detection unit 1032 detects the surroundings of a vehicle based on the data acquired by the sensor 101. Objects to be detected include, but are not limited to, for example, the number or positions of lanes, the number or positions of other vehicles around the vehicle, the number or positions of obstacles around the vehicle, for example, pedestrians, bicycles, structures, or buildings, the structure of roads, or road signs. Any object that is necessary for performing autonomous driving may be detected. Furthermore, the surroundings detection unit 1032 may track a detected object. For example, the surroundings detection unit 1032 may obtain a relative speed of an object from a difference between the coordinates of the object detected one step before and the current coordinates of the object. The data on surroundings detected by the surroundings detection unit 1032 (hereinafter, the surroundings data) is transmitted to the task control unit 1033 that will be described hereinbelow.

The task control unit 1033 controls traveling of a vehicle based on the operation plan generated by the operation plan generation unit 1031, the surroundings data generated by the surroundings detection unit 1032, and vehicle position information acquired by the position information acquisition unit 102. For example, the task control unit 1033 causes a vehicle to travel along a designated route such that obstacles do not enter a designated safety area around the vehicle. A well-known method may be employed as a method for performing autonomous driving of a vehicle. Furthermore, the task control unit 1033 may control operations other than traveling, for example, unloading, installing, or retrieving the storage apparatus 200 based on the operation plan generated by the operation plan generation unit 1031, and if necessary, based on the surroundings data generated by the surroundings detection unit 1032 or the vehicle position information acquired by the position information acquisition unit 102, etc.

The driving unit 104 drives the autonomous vehicle 100 based on the commands generated by the task control unit 1033. The driving unit 104 includes, for example, a motor for rotating the wheel, an inverter, a brake, a steering mechanism, and a secondary battery. The communication unit 105 is configured to connect the autonomous vehicle 100 to a network. In the present embodiment, the communication unit 105 may communicate with other apparatuses, for example, the server apparatus 300, through a network by using mobile communication services, such as 3G or LTE. Furthermore, the communication unit 105 may be further configured to communicate with another autonomous vehicle 100.

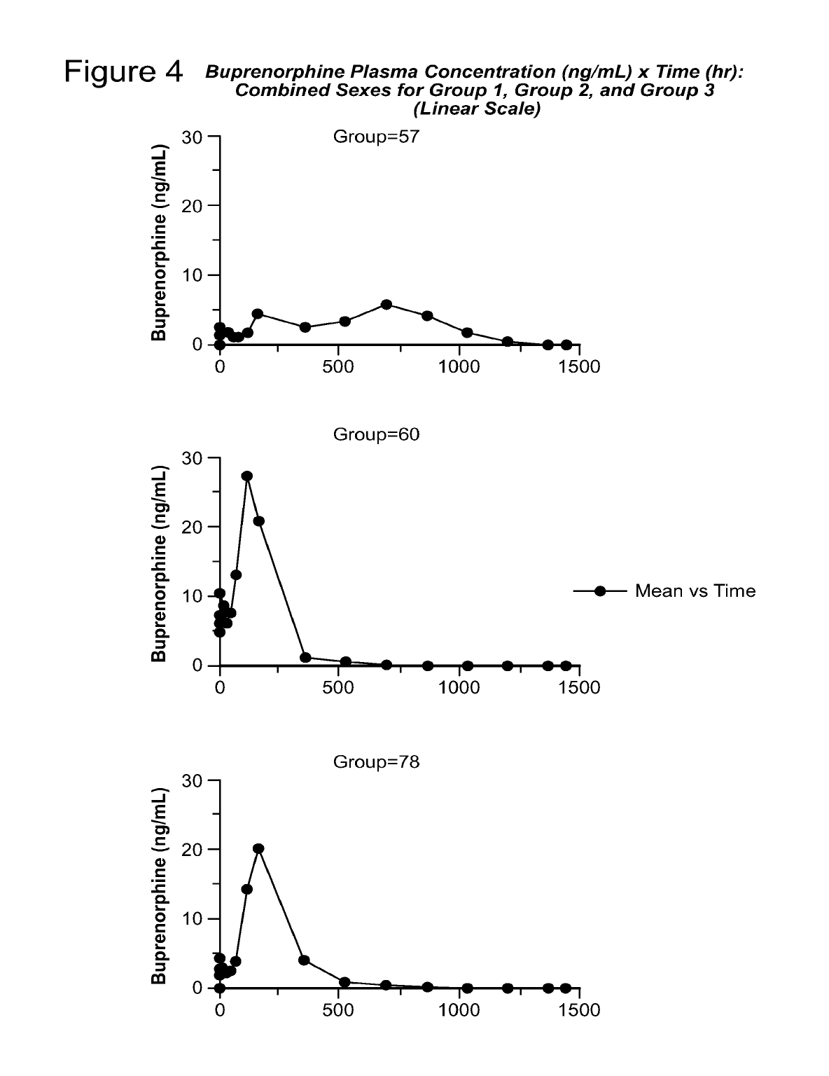

The autonomous vehicle 100 includes a loading unit 106, which is configured to load the storage apparatus 200. The autonomous vehicle 100 may load the storage apparatus 200 in a vehicle cabin, as illustrated in FIG. 4. Furthermore, in the example of FIG. 4, the front surface of the storage apparatus 200 faces outward. However, when the storage apparatus 200 is to be installed against a wall, the storage apparatus 200 may be loaded with the front and back side thereof reversed. FIG. 4 illustrates only one storage apparatus 200. However, the autonomous vehicle 100 may be configured to load a plurality of storage apparatuses 200. Furthermore, the autonomous vehicle 100 may be provided with a mechanism such as an elevator, an actuator, or a guide rail, for unloading or loading only a designated storage apparatus among the storage apparatuses 200. The above constituent elements are controlled by the task control unit 1033.

Next, the storage apparatus 200 is described. The storage apparatus 200 is a locker-type apparatus (a delivery locker) having a plurality of compartments each can store a package. As described above with reference to FIG. 2, the storage apparatus 200 is configured such that respective independent compartments may be accessed through a plurality of doors. The compartment for storing a package and a lock which is an electronic lock, are not illustrated in FIG. 3.

The storage apparatus 200 includes a communication unit 201, a lock control unit 202, a storage unit 203, and an input and output unit 204. The storage apparatus 200 is operated by power supplied from a battery which is not illustrated.

The communication unit 201, like the communication unit 105, is a communication interface for communicating with the server apparatus 300 via a network.

The lock control unit 202 is a computer that controls locking and unlocking of a plurality of compartments. The lock control unit 202 is, for example, a microcomputer. The lock control unit 202 may be implemented by executing a program stored in a storage, such as ROM, by a CPU (neither shown). The lock control unit 202 checks authentication information acquired from a recipient through the input and output unit 204 that will be described hereinbelow against authentication information previously stored in the storage unit 203 that will be described hereinbelow. The lock control unit 202 performs control of unlocking the electronic lock of a corresponding compartment when both pieces of authentication information match each other.

The storage unit 203 stores information and includes a storage medium, such as RAM, a magnetic disk, or a flash memory. Authentication information, which is checked when a package is handed over, is stored in the storage unit 203. The input and output unit 204 is an interface for providing a recipient with information and acquiring authentication information from the recipient. The input and output unit 204 includes, for example, a display device or a touch panel. Furthermore, the input and output unit 204 may be configured to acquire authentication information. For example, the input and output unit 204 may include a camera for reading a two-dimensional barcode, or may be configured to perform wireless transmission using near-field communication.

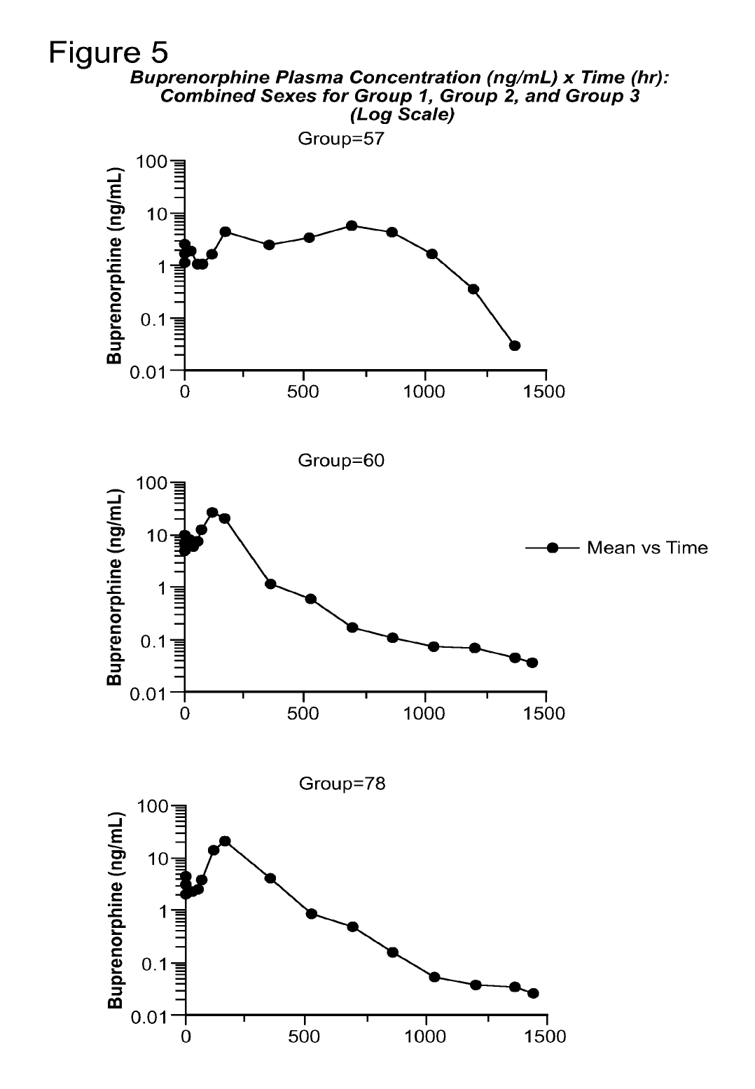

Also, the storage apparatus 200 illustrated in FIG. 2 is a stationary type. However, the storage apparatus 200 may be further configured for autonomous movement. For example, the storage apparatus 200 may further include a driving unit such as a wheel and a control unit configured to control the driving unit. With the above configuration, after being unloaded from the autonomous vehicle 100, the storage apparatus 200 may be autonomously arranged at a designated place. Furthermore, the storage apparatus 200 may be configured to fix the storage apparatus 200. For example, as illustrated in FIG. 5, the storage apparatus 200 may be connected, for example, inserted and fixed, to a lock 401, for example, an anchor, or a guide rail 402, which is fixed to the ground or a building, thereby preventing theft of the storage apparatus 200.

Next, the server apparatus 300 is described. The server apparatus 300 manages the positions and status of the autonomous vehicles 100, and generates and transmits an operation command. For example, when receiving, from a system manger, a request to deliver a storage apparatus 200 to a designated place, the server apparatus 300 acquires a destination and then transmits an operation command to the autonomous vehicle 100 that is available for delivery.

The server apparatus 300 includes a communication unit 301, a control unit 302, and a storage unit 303. The communication unit 301, like the communication unit 105, is a communication interface configured to perform communication with the autonomous vehicle 100 via a network.

The control unit 302 controls the server apparatus 300. The control unit 302 is, for example, a CPU. The control unit 302 has a vehicle information management unit 3021, an operation command generation unit 3022, and a package management unit 3023, as functional modules. Each functional module may be implemented by executing a program stored in a storage, such as ROM, by a CPU (neither shown).

The vehicle information management unit 3021 manages the plurality of autonomous vehicles 100. In detail, the vehicle information management unit 3021 acquires data on the autonomous vehicles 100 at each predetermined interval, from the autonomous vehicles 100, and stores the acquired data in the storage unit 303 that will be described hereinbelow. In the present embodiment, the vehicle information management unit 3021 acquires position information and vehicle information as the data on the autonomous vehicle 100. The vehicle information includes, but is not limited to, for example, an identifier, usage, and type of the vehicle, information on a holding area including a garage or a car sales office, a door type, a vehicle body size, a trunk size, a loading capacity, a drivable distance when fully charged, a drivable distance at a current time point, and a current status including the number, weight, volume, and delivery destination of loaded packages, of the autonomous vehicle 100.

When the server apparatus 300 receives an operation request of the autonomous vehicle 100, the operation command generation unit 3022 determines which autonomous vehicle 100 to send and generates an operation command responding to the operation request. The operation request includes, but is not limited to, for example, the following items:

(1) Delivery Request of the Storage Apparatus 200

This is a request for delivery of the storage apparatus 200 to a designated location. The request may include, for example, information on the number, size, weight and a delivery destination of the storage apparatus 200.

(2) Retrieval Request of the Storage Apparatus 200

This is a request for retrieving the storage apparatus 200 from a designated place. The request may include, for example, information on the number, size, weight, and a retrieval place of the storage apparatus 200.

An operation request may be issued by, for example, a system manager or a forwarding agent. In the following description, a subject who issues a request will be collectively referred to as a user.

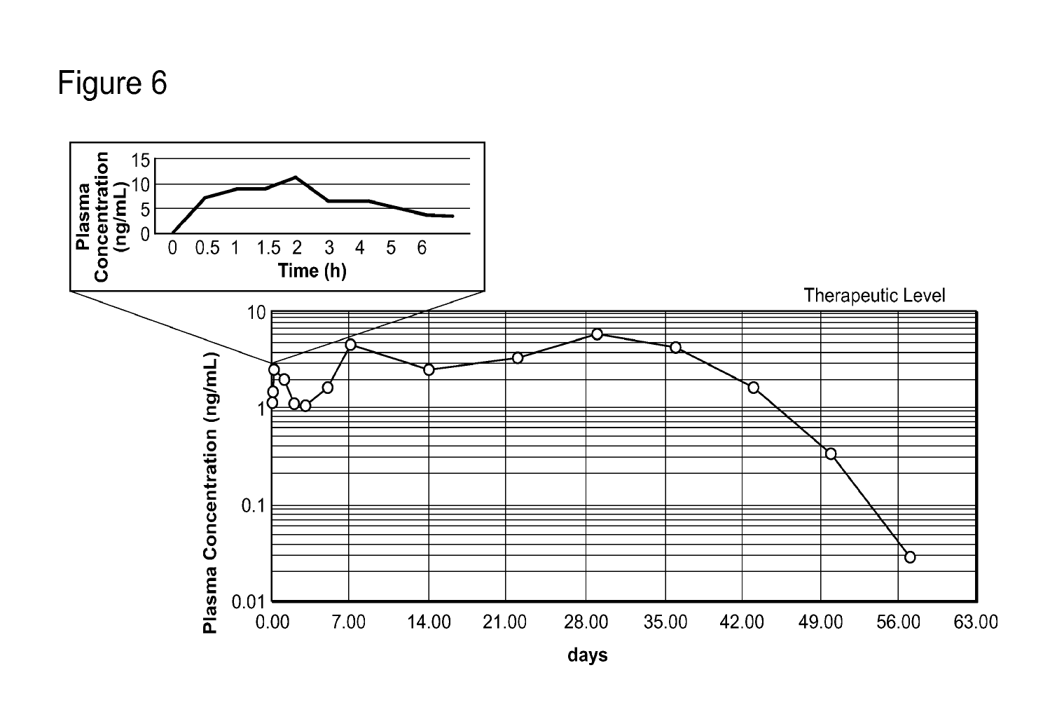

FIG. 6 illustrates an example of the operation command data generated based on the above information. The operation command includes type such as whether the command is for delivery or retrieval, an identifier of the storage apparatus 200 to be delivered, and an identifier of a place, that is a base, where the storage apparatus 200 is to be installed. Furthermore, other information may be included in the operation command.

The autonomous vehicle 100 that is to be a destination of the operation command is determined, for example, according to the vehicle position information and vehicle information on whether the vehicle is available for delivery or retrieval acquired by the vehicle information management unit 3021. Furthermore, the server apparatus 300, upon receiving an operation request, may immediately transmit an operation command to the autonomous vehicle 100 to move toward a destination. Furthermore, in order to receive a plurality of operation requests, the server apparatus 300 may transmit an operation command to the autonomous vehicle 100 to stand by for a designated period of time.

A package management unit 3023 manages information on unlocking a compartment of the storage apparatus 200. In detail, (1) when a package is stored, for example, by a courier, in one compartment of the storage apparatus 200, the package management unit 3023 generates first authentication information needed to unlock the compartment, and stores the first authentication information in the storage unit 203 of the storage apparatus 200. Furthermore, (2) the package management unit 3023 acquires information on a recipient, and transmits, to a terminal carried by the recipient, second authentication information needed to unlock the compartment which stores the package to be delivered to the recipient. In the present embodiment, the storage apparatus 200 is configured such that each compartment is capable of being locked by the electronic lock. When the first authentication information stored in the storage apparatus 200 matches the second authentication information acquired from the recipient, the storage apparatus 200 may unlock the compartment which stores the package to be delivered to the recipient. In other words, the package management unit 3023 generates each of the first authentication information and the second authentication information such that the two pieces of information match each other. Furthermore, an authentication method performed by the storage apparatus 200 may be a method of verifying identification by simply comparing pieces of authentication information or a method using an encryption key, for example, challenge-response authentication, or other well-known technologies.

The package management unit 3023, when storing of a package is completed, generates the first authentication information and the second authentication information, or selects the first authentication information and the second authentication information among a plurality of pieces of authentication information, and transmits the first authentication information to the storage apparatus 200. The first authentication information may be transmitted through wireless communication or may be input through the input and output unit 204. Furthermore, the package management unit 3023 is configured to acquire information to identify a terminal carried by the recipient, for example, an ID unique to an application or to an email address, and to transmit the second authentication information to the recipient's terminal when installation of the storage apparatus 200 is completed. The second authentication information may be a password or a two-dimensional barcode. Furthermore, when the terminal carried by the recipient and the storage apparatus 200 are able to perform near field wireless communication, the second authentication information may be transmitted wirelessly.

Also, the package management unit 3023 manages the plurality of storage apparatuses 200. In detail, the package management unit 3023 checks whether a package has been picked up from the storage apparatus 200, and if the pick-up status satisfies a designated condition, generates a trigger to allow the autonomous vehicle 100 to retrieve the storage apparatus 200. A detailed process thereof will be described hereinbelow.

The storage unit 303 stores information and is configured with a storage medium, such as RAM, a magnetic disc, or a flash memory. The storage unit 303 stores information to generate the first and second authentication information, information on a package to be delivered and the storage apparatus 200, information on a recipient, and the like.

Delivery of Storage Apparatus

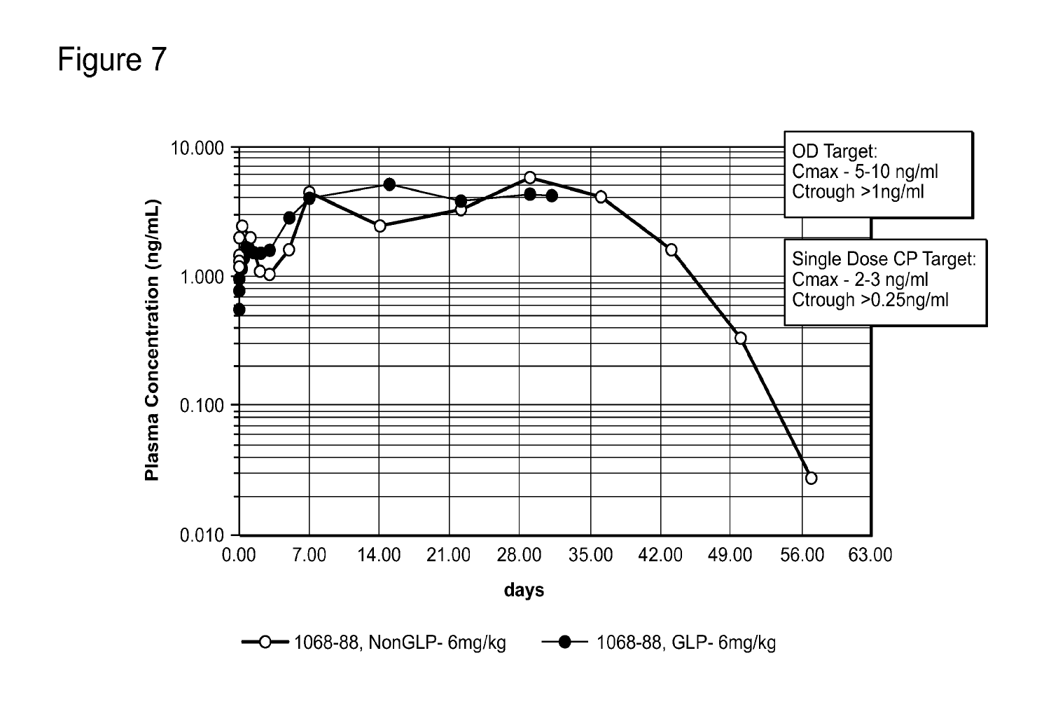

Next, the processes performed by the above-described respective constituent elements are described. FIG. 7 illustrates a data flow from the generation of an operation command by the server apparatus 300 based on an operation request acquired from a user 500 to the start of an operation by the autonomous vehicle 100.

The autonomous vehicle 100 periodically notifies the server apparatus 300 of position information. For example, when a road network is defined by nodes and links, the position information may be information specifying nodes or links. Furthermore, the position information may be coordinates such as latitude or longitude. The vehicle information management unit 3021 stores the notified position information in the storage unit 303 associated with the autonomous vehicle 100. Whenever the autonomous vehicle 100 moves, the position information is updated.

Also, the autonomous vehicle 100 periodically notifies the server apparatus 300 of vehicle information. In the present embodiment, the autonomous vehicle 100 transmits the following information as the vehicle information. Moreover, among the information listed below, information that is intrinsic to the autonomous vehicle 100 does not need to be repeatedly transmitted.

the capacity of a vehicle including loadable size, weight, or the number of storage apparatus

the number of storage apparatuses currently loaded

the volume of the storage apparatuses currently loaded

the weight of the storage apparatuses currently loaded

the current state of charge (SOC) of a battery

a travelable distance,

information on an operation route when the vehicle is presently in operation

information on storage apparatuses to be loaded on the autonomous vehicle 100 on an traveling route including the number, volume, weight, place, etc.,

information on storage apparatuses to be unloaded on the autonomous vehicle 100 on an traveling route including the number, volume, weight, place, etc.

Also, when the autonomous vehicle 100 is not in operation, for example, when the autonomous vehicle 100 is parked at a collection and delivery base, position information and vehicle information that are transmitted last are regarded as the latest information.

When the user 500 transmits an operation request to the server apparatus 300 through a communication unit, which is not shown (step S11), the operation command generation unit 3022 in the server apparatus 300 generates an operation command in response to the request (step S12).

In step S13, the operation command generation unit 3022 selects the autonomous vehicle 100 to provide a service. For example, the operation command generation unit 3022 refers to the stored position information and vehicle information on the autonomous vehicle 100 and determines which autonomous vehicle 100 is capable of providing the requested service. In step S14, the operation command is transmitted from the server apparatus 300 to the corresponding autonomous vehicle 100.

In step S15, the operation plan generation unit 1031 of the autonomous vehicle 100 generates an operation plan according to the received operation command. For example, the autonomous vehicle 100 generates an operation plan for performing loading or unloading of the storage apparatus 200, and for returning to a designated place, for example, the collection and delivery base, after completing the operation by specifying a traveling route, a place to load a storage apparatus 200, for example, a collection and delivery base, and a place to install the storage apparatus 200.

The generated operation plan is transmitted to the task control unit 1033, and an operation is started (step S16). Furthermore, during the operation, the position information and vehicle information are periodically transmitted to the server apparatus 300.

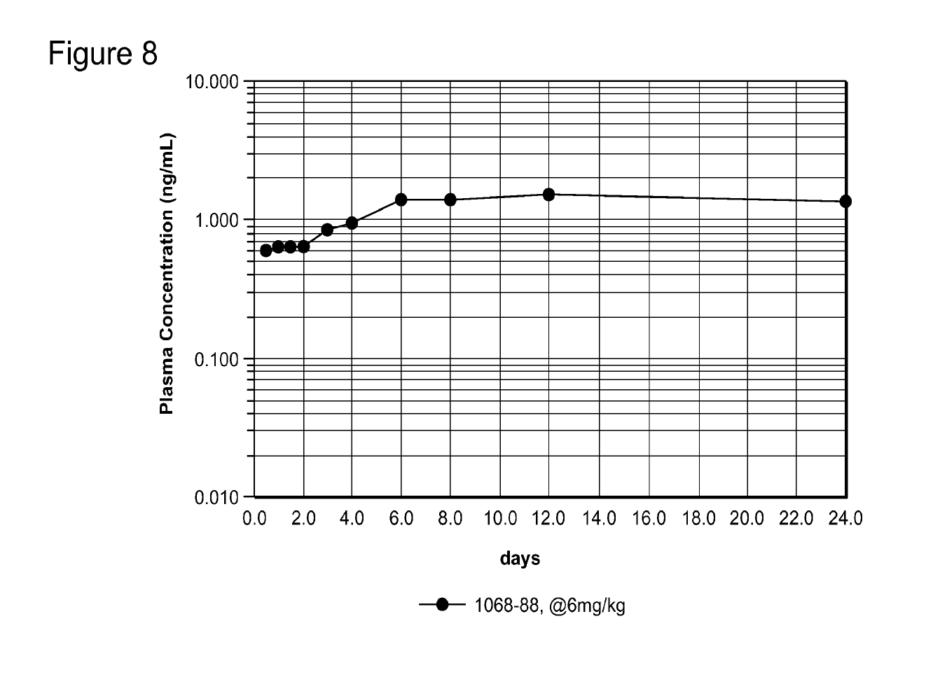

FIG. 8 is a flowchart of a process performed by the autonomous vehicle 100 after the operation is started in step S16. First, in step S21, the task control unit 1033 initiates traveling of the autonomous vehicle 100 toward the next destination, based on the generated operation plan. When the autonomous vehicle 100 has not yet completed loading of the storage apparatus 200, the next destination is a place where the storage apparatus 200 is loaded, for example, a collection and delivery base. On the other hand, when the autonomous vehicle 100 has completed loading of the storage apparatus 200, the next destination is a place where the storage apparatus 200 should be installed.

When the autonomous vehicle 100 approaches a target place (step S22), the task control unit 1033 searches for a place where the autonomous vehicle 100 can come to a stop in the vicinity, and stops the autonomous vehicle 100 and installs the storage apparatus 200 (step S23). The installation of the storage apparatus 200 may be autonomously performed or manually performed. For example, when the target place is a commercial facility, the task control unit 1033 transmits a message to a portable terminal carried by a person in charge of a store to call the person in charge to install the storage apparatus 200. In addition, the storage apparatus 200 may be configured to perform autonomous movement and automatically move from the vehicle.

When the storage apparatus 200 is newly installed, information to the that effect is transmitted as the vehicle information from the autonomous vehicle 100 to the server apparatus 300. In the present embodiment, the package management unit 3023 of the server apparatus 300, in response to the transmitted information, identifies a recipient corresponding to the storage apparatus 200 that has been installed, that is, a person who will receive the package stored in the storage apparatus 200, and transmits the corresponding second authentication information to the terminal carried by the recipient.

Next, the task control unit 1033 determines whether a next destination exists based on the operation plan (step S24). When there is a next destination, the task control unit 1033 controls the autonomous vehicle 100 to continuously perform the operation, and when there is no next destination, the task control unit 1033 controls the autonomous vehicle 100 to return to the collection and delivery base.

Retrieval of Storage Apparatus

When the recipient arrives at the location of the storage apparatus 200 installed at a designated place, the lock control unit 202 of the storage apparatus 200 acquires the second authentication information through the input and output unit 204, and checks the second authentication information against the first authentication information stored in the storage unit 203. The second authentication information may be acquired as text data through a keyboard or a touch panel, or as image data through a camera or a scanner. Furthermore, the second authentication information may be acquired through wireless communication. As a result, when the two pieces of authentication information match each other, the lock control unit 202 unlocks the compartment storing the package to be delivered to the recipient. Accordingly, the recipient may take out the package. When the package is taken out, the lock control unit 202 notifies the package management unit 3023 of the server apparatus 300 of the fact that the package has been taken out through the communication unit 201. Accordingly, the package management unit 3023 may recognize a pick-up status of packages in each storage apparatus 200.

Also, when a package has not been taken out after a designated period of time, the package management unit 3023 may transmit a reminder to the terminal carried by the corresponding recipient.

When the package pick-up status from the storage apparatus 200 satisfies a designated condition, the package management unit 3023 makes the operation command generation unit 3022 generate an operation command to make the autonomous vehicle 100 retrieve the storage apparatus 200. The storage apparatus 200 may be retrieved in the same sequence (see FIG. 7) and process flow (see FIG. 8) as when it was installed. The designated condition may be, for example, “when a designated time has passed after the storage apparatus 200 is installed,” or “when a designated number of packages have been taken out of the storage apparatus 200.”

As described above, according to the first embodiment, in the system for delivering a package using the autonomous vehicle 100, a delivery locker itself may be loaded and installed at a designated place. As such, convenience for the recipients may be improved, and costs of re-delivery may be reduced. In particular, since each of the storage apparatuses 200 is associated with a corresponding designated area, and the storage apparatus 200 is installed at the corresponding area, the recipient may receive a package near the recipient's residence.

### Second Embodiment

In the first embodiment, a package is handed over as the recipient operates the storage apparatus 200. However, according to the second embodiment, the storage apparatus 200 has an unmanned aerial object for delivering a package according to a recipient's request.

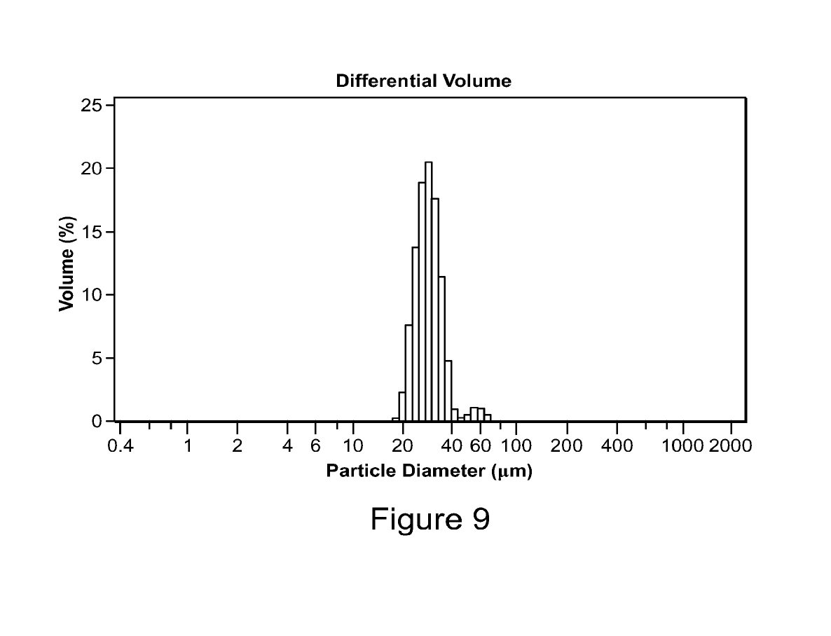

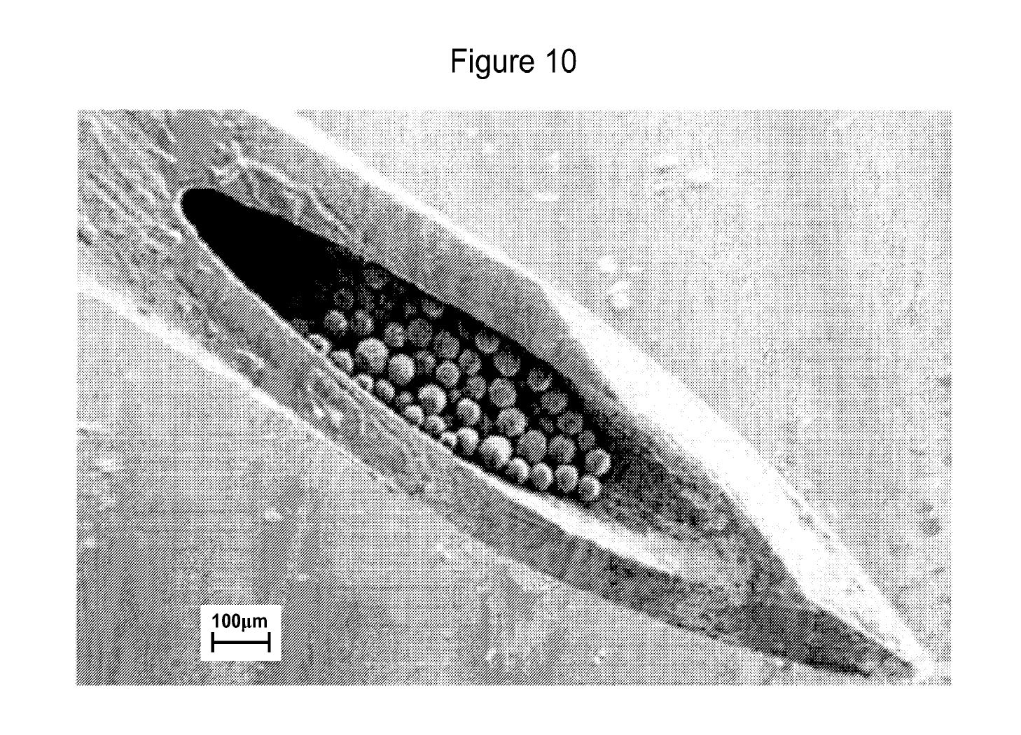

FIG. 9 illustrates the exterior of the storage apparatus 200 according to the second embodiment. As illustrated in the drawing, the storage apparatus 200 according to the second embodiment has a dual structure of an exterior case and an inner container. The storage apparatus 200 is accessible from the front surface thereof and be configured such that only the inner container is transportable. In addition, the inner container may be withdrawn only when authentication is completed using authentication information.

Also, in the second embodiment, the storage apparatus 200 includes an autonomous aerial object 901 for transporting the inner container. The autonomous aerial object 901 is an unmanned aerial object for transporting the inner container along a designated flight path, based on a command from the lock control unit 202.

In the second embodiment, the lock control unit 202 acquires the second authentication information through wireless communication. For example, the lock control unit 202 is connected to the terminal carried by the recipient and receives the second authentication information via a network. Accordingly, the recipient may perform authentication from a remote place. Furthermore, the lock control unit 202 stores flight path information corresponding to the recipient and, when the authentication is completed, transmits, to the autonomous aerial object 901, a command to transport the inner container for storing a package along the flight path. As such, the autonomous aerial object 901 may transport the inner container to a designated place.

A location to which the inner container is transported by the autonomous aerial object 901 may be any place that is near the storage apparatus 200. For example, a location to which the inner container is transported may be a balcony of a recipient's room, or the front area of an entrance of a recipient's room. The flight path may be set outside a building or follow a recipient's indoor movement. Information on a flight path may be previously stored in the storage unit 203 associated with the authentication information, and may be acquired from the terminal carried by the recipient. Furthermore, the autonomous aerial object 901 may include, for example, a sensor for autonomous flying.

According to the second embodiment, a package may be transported from the place where the storage apparatus 200 is installed to a recipient's residence. In particular, when the place where the storage apparatus 200 is installed is, for example, an entrance of an apartment complex, the recipient does not need to carry the package up to his or her residence, which greatly improves convenience for the recipients.

The above-described embodiments are merely examples, and appropriate modifications may be implemented within the technical scope of the disclosure.

For example, although in the description of the embodiment, a large storage apparatus 200 capable of storing a plurality of packages has been exemplified, the size of the storage apparatus 200 is not limited thereto. For example, a small storage apparatus capable of storing only one package may also be used as the storage apparatus 200.

What is claimed is:

1. An autonomous mobile object configured to transport a plurality of packages having recipients residing in a designated area, the autonomous mobile object comprising:
an operation controller configured to control the autonomous mobile object such that the autonomous mobile object autonomously travels to a designated place associated with the designated area;
a storage apparatus having a plurality of compartments, each compartment configured to be independently lockable and unlockable and store a package; and
a loading unit configured to load the storage apparatus storing the packages and to unload the storage apparatus at the designated place.

2. The autonomous mobile object according to claim 1, wherein the loading unit is configured to load a plurality of the storage apparatuses corresponding to a plurality of areas and to unload each of the plurality of storage apparatuses at a corresponding place.

3. The autonomous mobile object according to claim 1, wherein the storage apparatus is configured to be connected to a fixing unit configured to prevent movement of the storage apparatus from the designated place.

4. The autonomous mobile object according to claim 1, wherein the storage apparatus is configured to unlock, based on a result of authentication on a terminal associated with a recipient, the compartment associated with the recipient.

5. The autonomous mobile object according to claim 1, wherein the storage apparatus comprises an autonomous aerial object configured to transport, based on a result of authentication on a terminal associated with a recipient within a designated range, a package stored in the compartment associated with the recipient, in the designated range.

6. A delivery system configured to transport a plurality of packages having recipients residing in a designated area, the delivery system comprising:
a server apparatus; and
one or more autonomous mobile objects configured to perform transportation of a package,
wherein the autonomous mobile object comprises an operation controller configured to control the autonomous mobile object such that the autonomous mobile object autonomously travels based on a designated operation command, and a loading unit configured to load a storage apparatus storing the package and to unload the storage apparatus based on the operation command, and
wherein the server apparatus is configured to generate the operation command to make the autonomous mobile object travel to a designated place associated with the designated area and to make the loading unit unload the storage apparatus at the designated place, and transmit the operation command to the autonomous mobile object.

7. The delivery system according to claim 6,
wherein the loading unit is configured to load a plurality of the storage apparatuses corresponding to a plurality of areas, and
wherein the operation command comprises a command to make the loading unit unload each of the plurality of storage apparatuses at a corresponding place.

8. The delivery system according to claim 6,
wherein the storage apparatus comprises a plurality of compartments, each compartment configured to be independently lockable and unlockable, and
wherein the server apparatus is configured to store information on the recipient, and to transmit information to unlock a compartment corresponding to the recipient among the compartments of the storage apparatus to an apparatus associated with the recipient.

9. The delivery system according to claim 6, wherein the server apparatus is configured to acquire a pick-up status of a package stored in the storage apparatus, and when the pick-up status of the package satisfies a designated condition, to generate an operation command to make the autonomous mobile object retrieve the storage apparatus, and to transmit the generated operation command to the autonomous mobile object.

10. An autonomous mobile object configured to transport a plurality of packages having recipients residing in a designated area, the autonomous mobile object comprising:
an operation controller configured to control the autonomous mobile object such that the autonomous mobile object autonomously travels to a designated place associated with the designated area; and
a loading unit configured to load a storage apparatus having a plurality of compartments, each compartment configured to be independently lockable and unlockable and store a package, and to unload the storage apparatus at the designated place.

11. A server apparatus used in a delivery system configured to transport a plurality of packages having recipients residing in a designated area, the server apparatus comprising:
a processor configured to generate an operation command for one or more autonomous mobile objects transporting packages, and transmit the generated operation command,
wherein the autonomous mobile object includes an operation controller configured to control autonomous driving of the autonomous mobile object, and a loading unit configured to load or unload a storage apparatus storing the packages, and
wherein the operation command includes a command to make the autonomous mobile object travel to a designated place associated with the designated area and to make the loading unit unload the loaded storage apparatus at the designated place.

12. The server apparatus according to claim 11,
wherein the loading unit is configured to load a plurality of the storage apparatuses corresponding to a plurality of areas, and
wherein the operation command includes a command to make the loading unit unload each of the plurality of storage apparatuses at a corresponding place.

13. The server apparatus according to claim 11,
wherein the storage apparatus is configured to have a plurality of compartments, each compartment configured to be independently lockable and unlockable, and
wherein the server apparatus is configured to store information on a recipient, and to transmit information to unlock a compartment corresponding to the recipient among the compartments of the storage apparatus to an apparatus associated with the recipient.

14. The server apparatus according to claim 11, wherein the processor is configured to acquire a pick-up status of a package stored in the storage apparatus, and when the pick-up status of the package satisfies a designated condition, to generate an operation command to make the autonomous mobile object retrieve the storage apparatus.
